(19) United States
(12) Patent Application Publication
(10) Pub. No.: US 2008/0011301 A1
(43) Pub. Date: Jan. 17, 2008

(54) Out flow resistance switching ventilator and its core methods

(76) Inventor: Yuancheng Qian, Glen Allen, VA (US)

Correspondence Address: Yuancheng Qian, 4501 Sadler Grove Ct, Glen Allen, VA, 23060, US

(21) Appl. No.: 11/484,313

(22) Filed: Jul. 12, 2006

(51) Int. Cl. A61M 16/00

(52) U.S. Cl. 128/204.23; 128/204.21

(57) ABSTRACT

An out flow resistance switching ventilator and its methodology for providing mechanical ventilation support to the respiratory failure patient are claimed. Based on the continuous out flow impounding ventilation mechanism, the apparatus provides ventilation by switching flow resistance between two different levels at a continuous gas flow system outlet valve controlled by patient's spontaneous breathing or preset mandatory parameters, resulting in airway pressure levels switching, and therefore creating lung volume switching, which is totally different from either volume ventilation or pressure ventilation mechanism utilized in the current conventional ventilators.

### CROSS-REFERENCES

1. Pingleton, S. K. Complications of acute respiratory failure. American Review of Respiratory Diseases. 1988, 137: 1463-1493.
2. Dreyfuss, D and Saumon, G. Ventilator-induced lung injury. American Journal of Respiratory and Critical Care Medicine. 1999, 157: 294-323.
3. Brochard, L. Chapter 9, Pressure support ventilation. In M. J. Tobin, editor. Principles and practice of mechanical ventilation. McGraw-Hill, New York. 1994. 239-257.
4. Puritan Bennett Company. Pressure control ventilation review.
5. Puritan Bennett Company. Bilevel: two ventilation strategies in one mode. 2002.
6. Nellcor Puritan Bennett Company: Bilevel option/800 series ventilator, Operator's and technical reference manual of 840 ventilator system. 2002.
7. Respironics Inc. The BiPAP S/T clinical manual. 1996.

### STATEMENT REGARDING FEDERALLY SPONSORED RESEARCH OR DEVELOPMENT

Not Applicable

### BACKGROUND OF THE INVENTION

As a life support device, ventilator is one of the key equipments in the modern critical and emergency medicine by providing ventilation and oxygenation support to the respiratory failure patient.

The conventional ventilators work on two different basic forms of mechanism, i.e. volume ventilation and pressure ventilation. These mainstream ventilators provide tidal volume by driving positive pressure gas flow into the airway and the lung. Ventilator generates and drives pressure gas flow periodically. Since patient's breathing is periodical, and his/her airway and lung is such a complicated tubing—air space system, the pressured gas delivery pattern must match with the resistance and compliance changes in the airway and the lung, as well as with the patient's needs for inspiratory flow at several manners, such as initiation, flow rate, timing, volume, and pressure, etc. Any improper setting at these aspects may cause patient's discomfort, high airway pressure, even fighting with ventilator.

These disadvantages are quite apparent when volume ventilation is used in the patients with severe pathologic changes in airway or lung, because the flow in volume ventilation is preset and not dependent on the airway conditions and patient's inspiratory needs. It is already addressed very clearly, that high airway pressure is the major cause for ventilator induced lung injury. Therefore, the attempts to improve safety, efficiency and comfort have been the focus of clinical ventilation support and ventilator technology, and the major purpose and consequence of all these tries are to lower the airway pressure.

During the last two decades the introduction of pressure ventilation modes, including pressure support ventilation and pressure control ventilation, and their widespread use have been a very important progress of mechanical ventilation. Pressure ventilation's better ability to change its flow pattern to meet patient's inspiratory needs at every moment makes airway pressure lower than otherwise in volume ventilation, especially in complicated pathologic airway situations. But still, it is very difficult to totally avoid the mismatch between flow output and patient's needs when inspiratory pressured flow is being forced to move into the airway, since pressure ventilation is also based on the inspiratory positive pressure flow generating and driving mechanism.

In the last few years, a new mode named Bi-level has been equipped on some advanced ventilators. Its performance over the mainstream ventilation forms has already been shown by two basic and impressive facts: Bi-level can much easier and quicker slow down the respiratory distress patients without sedation, and Bi-level can significantly lower airway pressure to achieve the same inflation volume level as with pressure support or pressure control ventilation. The inventor addresses the working mechanism of Bi-level as APPSV, the airway positive pressure switching ventilation.

APPSV, the airway positive pressure switching ventilations, is a new and totally different form from the conventional ventilation, and it works on the continuous flow impounding mechanism. The APPSV gas source supplies a fixed rate continuous flow into system, as the gas flow is impounded by resistance created on the outlet valve in the unit circuit, the positive pressure in the whole system including airway and lung is created. Since the lung works as an elastic chamber, the change of lung volume always follows the change of the pressure in lung. The relationship between lung volume and inside pressure is expressed as the lung pressure—volume curve. Different level of lung and airway pressure represents a different lung volume. At APPSV, airway pressure is switched at two different levels, lung volume then is switched at two levels correspondingly, and this means ventilation.

The picture of APPSV is just like what happens in a reservoir, the closing of the sluice gate at the dam raises the water level at upstream. In APPSV the lung is inflated not because of inspiratory pressure flow forced to move into the airway but because of pressure rising in the whole system. By this manner, there are no longer matching problems between flow driving pattern and patient inspiration needs. The APPSV's outstanding property has been shown by Bi-level's performance.

So far Bi-level has been only used as a non-primary mode in the mainstream ventilators, in which conventional volume or pressure ventilation mechanisms are still utilized to provide basic ventilation support. Technically, in current Bi-level mode, airway pressure switching is still upon the preset timing parameters and unable to be led by patient's spontaneous breathing, therefore it still does not totally meet the patient's need at some aspects, such as ventilation rate and inflation to deflation phase ratio.

Based on the performance of Bi-level, the inventor believes that a new ventilator totally working on APPSV mechanism will be a very significant development in ventilator technology and might be a new direction for the mechanical ventilation support.

### BRIEF SUMMARY OF THE INVENTION

An out flow resistance switching ventilator is designed to convert the abstract principle of APPSV into a true life saving apparatus to meet two basic requirements for clinical ventilation support: to provide patient's spontaneous breathing more space to lead ventilation support and to guarantee the ventilation safety at any desired level.

As the core part of the invention, a dual solenoids electromagnetic outlet valve is developed. By creating and switching of out flow resistance at outlet valve in the continuous flow system, two levels of system and airway pressure and lung volume are created and switched, providing oxygenation and ventilation effects.

Flow resistance or airway pressure switching modes are designed to be controlled by patient's spontaneous breathing, machine mandatory preset parameters or spontaneous—mandatory combination to meet the different needs in different clinical situation. Also, utilizing a microchip performing a programmed ventilation pressure adjusting processing, the target volume function is designed to keep ventilation volume at preset level automatically.

The out flow resistance switching ventilator and all associated designs and techniques listed above are not found in any previous application, report or patent claim.

### DETAILED DESCRIPTION OF THE INVENTION

As set forth in claim 1, the ventilator, which works on the base of flow resistance switching ventilation mechanism, is claimed.

The ventilator supplies a continuous gas flow at 120 LPM through the circuit system, and creates the positive pressures switching in system by flow resistance switching as the flow passes the dual solenoids electromagnetic outlet valve setting at the end of the from patient limb of the unit circuit. Since lung volume is corresponding with the system and airway pressure, the system pressure switching creates the lung volume switching, i.e. ventilation.

Due to the out flow resistance switching, two levels of pressure in system including airway and lung pressures are created, and these two parameters are the key factors determining the ventilation support: the base level of airway pressure determines the residual lung volume, relating the oxygenation; and the ventilation pressure determines the ventilation volume. On the pressure waveforms, ventilation pressure plus the base level of airway or lung pressure, i.e. airway or lung deflation pressure, equals the airway or lung inflation pressure.

As the necessary functions of a modern ventilator, spontaneous ventilation mode and spontaneous assist—mechanical mandatory mode are designed to provide patient's spontaneous breathing the full space to lead ventilation support and to guarantee the patient to receive the minimum ventilation. In addition, for patient's ventilation safety, apnea ventilation is also designed to provide rescue ventilation when no spontaneous breathing or mandatory ventilation determined within a preset apnea alarm period.

The target volume function is designed to provide an option to keep patient's ventilation volume at a preset level, utilizing computer control technique to adjust the ventilation pressure automatically, instead of adjusting the ventilation pressure manually.

Through all these major ideas and designs, a real life saving ventilator is developed upon the simple principle of airway pressure switching ventilation mechanism.

As set forth in claim 2, the outlet valve setting at the end of the from patient limb of unit circuit is the core part of the ventilator to create and control the pressure in system by its dual solenoids electromagnetic threshold resistor design.

As the electromagnetic threshold resistor, the magnetic field is generated as electric current passing through the solenoids and pushes the valve's actuating shaft and disk against the outward continuous flow passing the valve outlet, resulting in the creating of the positive pressure in system and airway.

In the valve I designed, one solenoid is always carrying with a non-intermittent current to create the base level of system pressure.

While the other solenoid is assigned to carry an intermittent current and generate the field force responsible for ventilation pressure. The status of intermittent current is controlled by the flow processing chip, the timing processing chip, or the apnea ventilation processing chip. As the current is switched on by spontaneous effort or mandatory timing, the forces generated by the solenoids will push on the valve disk and creates a ventilation pressure in system and airway, making a higher inflation pressure over the base level. As the intermittent current is ceased, only the force generated in the solenoid carrying non-intermittent current works on the disk, the pressure in system and airway then goes back to the lower base level.

In short, as the core part to provide ventilation support, the switching of the current status in the solenoid with intermittent current determines ventilation's pattern, rate and interval; while the amount of the intermittent current determines the ventilation pressure, correspondingly, ventilation volume; and the amount of the non-intermittent current determines the base airway pressure, functional residual volume, correspondingly. All these primary ventilation support parameters are created on this single valve.

In older to turn the APPSV method into a real functional apparatus, three different ventilation modes are designed in this apparatus to guide the switching between airway pressure levels and to meet all requirements for a ventilator used in clinic. The idea and design of the switching between airway pressure levels is key for the invention. None of these three ideas and designs have ever been claimed by any other inventor before.

In this apparatus, the ventilation modes, i.e. the initiating and stopping of the intermittent current on the solenoid responsible for the ventilation pressure, is controlled by three micro-processing chips organized at three levels.

As the top one, the flow processing chip delivers signals to the solenoid to guide the spontaneous ventilation, meanwhile the signals are delivered to the timing processing chip to start timing counting for a mandatory ventilation and to the apnea ventilation processing chip to start timing counting for a apnea alarm and rescue ventilation;

The timing processing chip is the middle one and controls mandatory ventilation issue. The processing chip receives the signal from the flow processing chip and starts a preset mandatory ventilation period timing counting. If there is no next signal received till the end of preset mandatory ventilation period, a signal will be delivered to the solenoid for the ventilation pressure to initial a cycle of mandatory ventilation, at same time a signal is delivered to the apnea ventilation processing chip to start timing counting for apnea alarm and ventilation;

The apnea ventilation processing chip receives signals from the flow processing chip and the timing processing chip and starts a preset apnea alarm period timing counting; if there is no signal received either from the flow chip or from the timing chip till the end of preset apnea alarm period, as the one at the lowest level, the apnea ventilation processing chip will directly deliver a signal to the solenoid to initiate a rescue ventilation in order to guarantee the patient's ventilation safety.

Spontaneous ventilation (SV) mode is the most important feature in the invention. The vast majority of patients need ventilation support not because of impaired breathing driving stability, but because of the fatigue or weakness of respiratory muscles and because of reduced residual lung volume. For this group of patients with quite stable spontaneous breathing driving, what ventilation supprt should provide is the inspiratory power, not the guarantee of ventilation rate stability. It is very important for this group during ventilation support to let patients' spontaneous breathing guide mechanical ventilation.

As set forth in claim 3, patient spontaneous breathing is allowed to lead the out flow resistance switching ventilation, or airway pressure switching ventilation. Therefore, identifying the start and the end of spontaneous inspiration as the switching points of system and airway pressure is the key technique for SV mode, and this is conducted by the flow processing chip.

Since the patient's circuit is a continuous flow system, the flow rates in the to patient limb and the from patient limb are equal when there is no gas flow moving in the airway between two cycles of breathing. As gas flow starts to enter the airway due to patient's inspiratory effort, the flow in the from patient limb will start being smaller than what in the to patient limb. Receiving the flow signals from the two gas flow sensors located at said two limbs, the flow processing chip determines it as the beginning of the spontaneous inspiration and sends the signal to the solenoid responsible for the ventilation pressure to switch on the intermittent current. The ventilation pressure then is created on the outlet valve.

While at the end of inspiration, since there is no gas movement in the airway, no flow difference in two circuit limbs will be detected by the flow processing chip and the signal to cease the current will then be sent. Since ventilation pressure is no longer added on the valve, pressure created in system and airway will go back to base level. The method of patient spontaneous assist—preset timing mandatory (SMV) mode is developed as set forth in claim 4.

For the group of patients with unstable spontaneous breathing driving or patients with special reason for certain level hyperventilation, SMV mode can guarantee the patient's minimum ventilation rate. The idea of SMV is to allow patient to breathe spontaneously all the time and to guarantee patient to be ventilated with a minimum rate.

At SMV mode, ventilation cycle, i.e. the switching of status of the intermittent current on the solenoid is designed to be controlled by both the flow processing chip and the timing processing chip.

The timing processing chip is the control part to conduct SMV mode. It starts to preset mandatory ventilation period time counting at every moment when the intermittent current on the solenoid is switched on.

If there is no spontaneous effort within a preset mandatory ventilation period, timing processing chip will deliver a signal to the solenoid to switch the current on, i.e. to initiate a cycle of mandatory ventilation.

Since the flow rates in the two limbs are unequal before the end of deflation of this mandatory ventilation, no spontaneous ventilation will be initiated if any spontaneous effort occurs. Because according to the design, the flow processing chip switches on the intermittent current to initiate a cycle of ventilation only when the equal flow rates in the two limbs start to become unequal. But this spontaneous effort still can inhale gas flow from the circuit with no limitation.

In most cases, no matter spontaneous breathing is faster or slower than the preset mandatory rate, as long as a spontaneous effort occurs when the flow rates are equal in the two limbs, a cycle of spontaneous ventilation will be initiated.

As a life support device, a ventilator must have the function to initiate the visual and audible alarms, and to provide the properly preset rescue ventilation to the patient with no breathing or not being ventilated for a preset apnea alarm period, Automatically. The method of apnea alarm and apnea ventilation (AV) is developed as set forth in claim 5.

The apnea ventilation processing chip is the core part which is in change of the apnea alarm ventilation issue and is operating all the time. The apnea ventilation processing chip receives the signal from the flow processing chip indicating the beginning of a cycle of spontaneous ventilation, or the signal from the timing processing chip indicating the beginning of a cycle of mandatory ventilation. Once a signal is sent to the apnea ventilation processing chip, a preset alarm period time counting starts; and if no next signal arrives before the preset apnea alarm period elapses, the intermittent current in the solenoid for ventilation will be initiated and stopped by the preset parameters repeatedly until the next signal arrives before the apnea alarm period elapses.

In this ventilator, the target volume technique is developed to keep ventilation volume at a preset level as set forth in claim 6.

At APPSV, the ventilation pressure i.e. the difference between the inflation pressure and the deflation pressure is the primary factor to determine the ventilation volume; but the ventilation volume is also affected by the other factor, the chest and lung compliance. Because the compliance is uncertain, there is no fixed relationship between the volume and pressure, therefore for ASPPV ventilation, it is impossible to set a ventilation volume directly or to set a ventilation pressure leading to a certain ventilation volume. During APPSV, repeatedly trying and adjusting the ventilation pressure to make ventilation volume close to a desired level is a usual procedure. This is the essential characteristic of APPSV.

As set forth in claim 6, the target volume function is designed to provide a direct way to reach a preset ventilation volume level by setting a target volume. This function does not change the characteristics of ASPPV, just utilizes the computer technique to do the job, which is usually done by the manual.

In my design, the target volume chip is assigned to calculate the ventilation pressure needed to get the preset target volume according to data received from the date monitoring chip, including previous ventilation pressure used and actual ventilation volume achieved.

After the target volume function and value is selected and started, a preset 10 cm H20 of testing ventilation pressure is used to initiate the first ventilation by the target volume processing chip automatically; after this cycle of ventilation, the data of measured volume and ventilation pressure are sent to the chip; the ventilation pressure needed to reach the target volume in this circumstance is calculated; this calculated value will be set by the chip for the next cycle of ventilation to try to achieve the target volume.

Same process is conducted repeatedly in each cycle of ventilation, to adjust or keep the volume patient actually receive very close to preset target volume value.

I claim:

1. An out flow resistance switching ventilator to provide ventilation and oxygenation support for the respiratory failure patients totally based on the method of out flow resistance switching at two different levels comprising the steps of:
supplying a continuous gas flow into unit circuit at a fixed high rate;
utilizing a dual solenoids electromagnetic outlet valve setting at the end of from patient limb of unit circuit to create and switch the flow resistance at two different levels, therefore two different levels of positive pressure in system including airway and lung created and switched, and resulting in the switching of two different levels of lung volume, i.e. resulting in ventilation;
through two ventilation modes designed, allowing patient's spontaneous breathing to guide the switching of gas flow resistance to provide patient the maximum space to lead ventilator's work, or if necessary, allowing the preset mandatory timing cycle to control the switching of gas flow resistance to guarantee patient the minimum ventilation level;
initiating the rescue ventilation automatically once no gas movement detected in airway for a preset apnea alarm period to guarantee patient's ventilation safety, as a requisite function of a life saving device.

2. The method and system of dual solenoids electromagnetic outlet valve to create and control the system pressure comprising the steps of:
setting at the end of the from patient limb of unit circuit to impound systemic gas flow and to create positive pressure in whole system including airway and lung as set forth in claim 1;
as an electromagnetic threshold resistor, the magnetic field generated as electric current passing through the solenoids of said valve, pushing the valve's actuating shaft and disk against the outward continuous flow passing the valve outlet, creating positive pressure in the system including airway and lung; the field force intensity and created system pressure determined by the strength of electric current;
dual solenoids designed to create two pressure levels separately;
one solenoid always carrying with a non-intermittent current to generate the field force responsible for the base level of system pressure;
the other solenoid with the intermittent current assigned to generate the field force responsible for ventilation pressure;
force generated as said intermittent current switched on by patient's spontaneous effort or mandatory timing, creating the ventilation pressure in system, added over the base level of system pressure;
while said intermittent current ceases, only the force generated on the solenoid with non-intermittent current works on the disk, the system pressure then goes back to the base level;
both base system pressure and ventilation pressure adjusted by setting of the strength of the electric currents.

3. The method of spontaneous ventilation (SV) consists of the steps of:
referred to as SV mode, patient's spontaneous breathing allowed to guide the switching of the intermittent current status on the solenoid responsible for ventilation pressure, resulting in the system including airway and lung pressure switching between the lower level or base pressure, or deflation pressure, and the higher level, or inflation pressure, which equals base pressure plus ventilation pressure;
as the control part of SV mode, three jobs conducted by the flow processing chip: to determine patient's breathing status according to the signals transmitted from the flow sensors, to control the intermittent current status on the solenoid responsible for ventilation pressure, and to send the signal to timing processing chip to start a preset mandatory period timing counting and the signal to the apnea ventilation chip to start a preset apnea alarm period timing counting;
two gas flow sensors setting at the to patient limb and the from patient limb separately used to measure flow rates in each said limbs;
flow signals from said two different sensors transmitted into the flow processing chip for identifying the status of each cycle of patient spontaneous breathing:
as the gas flow starts to enter airway due to spontaneous inspiratory effort, the flow difference between larger flow in the to patient limb and smaller flow in the from patient limb identified as the beginning point of a cycle by the flow processing chip, the signal then delivered by the flow processing chip to switch on the intermittent current in the solenoid responsible for the ventilation pressure, as set forth in claim 2, the ventilation pressure is therefore created, resulting in lung volume increasing and the inflation;
at the end of inflation, since no gas flow moving into the airway any more, the equal flow rates in the to patient limb and the from patient limb determined by the flow processing chip, the current in solenoid responsible for the ventilation pressure then ceased by the flow processing chip, and the ventilation pressure no longer created on the outlet valve, as set forth in claim 2, the system including airway and lung pressure back to the base level, resulting in the lung volume decreasing and the deflation.

4. The method of patient spontaneous assist—preset timing mandatory (SMV) mode for switching of system including airway and lung pressures comprising the steps of:
to initiate and cycle mandatory ventilation by the preset timing parameters to guarantee patient's minimum ventilation rate once a preset mandatory ventilation period elapsed; meanwhile still to allow patient spontaneous breathing guide ventilation as long as its inspiratory effort occurs after the end of deflation, therefore referred to as SMV mode in this machine;
in SMV mode, ventilation, i.e. the switching of the intermittent current in the solenoid responsible for the ventilation pressure, controlled by both the timing processing chip and the flow processing chip;
as the primary controller of SMV, two major features conducted by the timing processing chip: to start timing counting for a preset mandatory ventilation period at an initiating point of a cycle of mandatory ventilation or an initiating point of cycle of spontaneous ventilation, marked by the initiating of intermittent current in the solenoid responsible for ventilation pressure; to initiate a cycle of switching of intermittent current in the solenoid responsible for ventilation pressure by the preset rate and interval, i.e. to provide a cycle of mandatory ventilation if a said preset mandatory ventilation period elapses;
in SMV mode, patient's spontaneous breathing allowed all the time; but by the rule of the flow processing chip, only when flow in the from patient limb is equal to flow in the to patient limb, a spontaneous inspiratory effort allowed to switch on the intermittent current in the solenoid responsible for the ventilation pressure, i.e. only when the expiration or deflation ends, a cycle of spontaneous ventilation allowed to be initiated, as set forth in the claim;
any spontaneous inspiratory effort before the end of deflation not allowed to initiate the ventilation because it does not meet the chip's rule to switch on the intermittent current in the solenoid responsible for the ventilation, but gas flow still allowed to be inhaled into airway freely;
if no spontaneous breath effort determined as a preset mandatory ventilation period elapsed, the intermittent current in the solenoid responsible for the ventilation pressure switched on and off by preset rate and interval, i.e. a cycle of mandatory ventilation initiated and cycled conducted by the timing processing chip;
time counting for a preset mandatory ventilation period repeated from every moment as the intermittent current in the solenoid responsible for the ventilation switching on, either spontaneous ventilation or mandatory ventilation goes on by the rules described above.

5. The method of the apnea ventilation comprising the steps of:
providing the properly preset rescue ventilation (AV) to the patient once lacking of spontaneous breathing or not being ventilated in the preset apnea alarm period, to meet an essential requirement for a ventilator as a life support device;
as a unique design in this machine, ventilation support is not to be started until all apnea ventilation parameters including apnea alarm set up and confirmed;
as the controller of AV mode, two features conducted by the apnea ventilation processing chip: to start a preset apnea alarm period time counting as receiving a signal indicating the initiating point of intermittent current in the solenoid either from the flow processing chip or from the timing processing chip, i.e. to start counting a preset apnea alarm period after a cycle of spontaneous ventilation or a cycle of the mandatory ventilation initiated; once no signal received by the end of a preset apnea period, to initiate the apnea alarm and a cycle of starting and stopping the intermittent current in the solenoid responsible for the ventilation pressure by preset apnea ventilation rate and the interval, i.e. to provide a cycle of rescue ventilation;
the apnea ventilation processing chip working all the time either SV or SMV selected.

6. The method of target volume function comprising the steps of:
utilizing computer technique to provide the function for a direct way to achieve a selected ventilation volume by calculating and adjusting the ventilation pressure, conducted by the target volume processing chip;
as a ventilation pressure controller, two features conducted by the target volume processing chip: to calculate the ventilation pressure needed to achieve the target volume according to the previous ventilation data feeded back from the system data monitoring chip; to send signal to adjust the amount of current in the solenoid responsible for ventilation pressure to get said calculated pressure;
the formula used to calculate the ventilation pressure (VP) needed to achieve the target volume based on the ventilation pressure applied and the volume actually achieved:
VP needed/target volume=VP applied/volume achieved;
as the initial ventilation, after target volume function and target volume selected and confirmed, a 10 cmH2O of test ventilation pressure applied to inflate the lung by the target volume processing chip, and the corresponding inflated volume measured by monitoring system and feeded back to the said chip, then the VP needed to achieve the set target volume calculated by the said chip by applying data received into the formula:
VP needed=10 cmH2O×target volume/volume measured;
said value of VP needed set by the target volume processing chip to inflate the lung for second cycle of ventilation to try to achieve the target volume, the ventilation data feedback and value calculation for VP needed conducted again in the same way as after the initial ventilation;
ventilating and modifying repeatedly to keep or adjust ventilation volume at the level very close to the target value.
